(19) United States
(12) Patent Application Publication
(10) Pub. No.: US 2016/0090336 A1
(43) Pub. Date: Mar. 31, 2016

(54) REMOVAL OF AROMATIC CONTAMINANTS IN OLEFIN STREAM FROM PARAFFIN DEHYDROGENATION

(71) Applicant: UOP LLC, Des Plaines, IL (US)

(72) Inventor: Robert L. Mehlberg, Wheaton, IL (US)

(21) Appl. No.: 14/496,086

(22) Filed: Sep. 25, 2014

(51) Int. Cl. C07C 5/333; C07C 7/10; C07C 7/04; C07C 7/12; C07C 7/13

(52) U.S. Cl. CPC C07C 5/333; C07C 7/12; C07C 7/13; C07C 7/04; C07C 7/10

(57) ABSTRACT

A process is presented for the removal of aromatics from an olefin stream. The process includes washing the olefin stream with a hydrocarbon stream to absorb aromatics generated in a dehydrogenation process. The olefin stream is compressed, then washed and cooled with a liquid hydrocarbon stream. The cooled and washed stream is passed to a separation drum to remove the liquid hydrocarbon stream containing the aromatics.

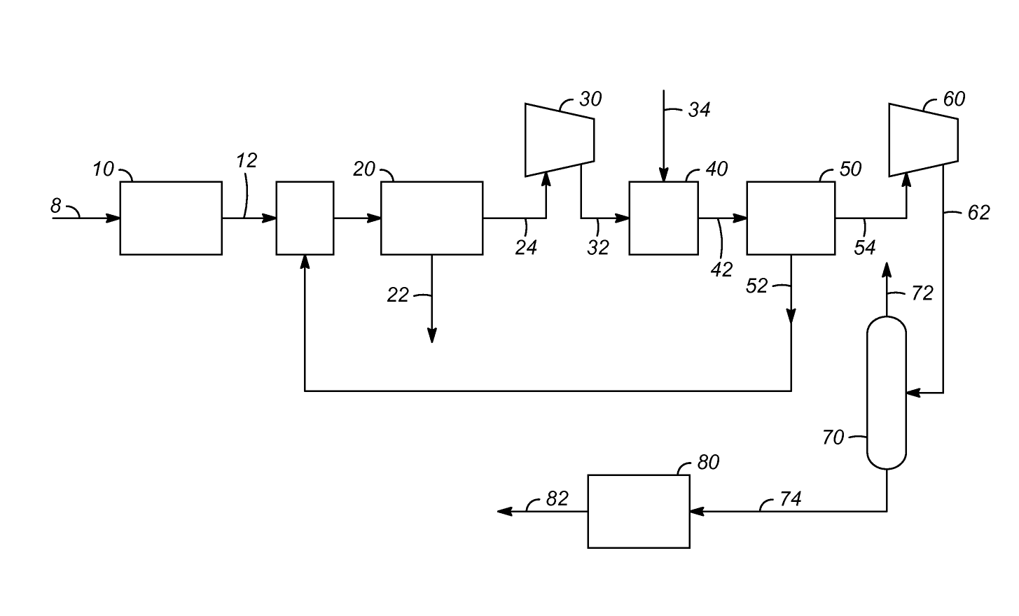

### FIELD OF THE INVENTION

The field of the invention relates to paraffin dehydrogenation in the production of olefins. In particular, the invention relates to removing contaminants from an olefin generated feedstock.

### BACKGROUND

Currently, the majority of light olefins are produced from cracking processes, either stream or catalytic cracking. Cracking is the process of breaking larger hydrocarbon molecules into smaller hydrocarbon molecules through contacting the larger hydrocarbon molecules with a catalyst at reaction conditions. The catalytic cracking process is one method used to produce ethylene and propylene from hydrocarbon feedstocks. The ethylene and propylene are important chemicals for the production of the respective plastics polyethylene and polypropylene, two important plastics having a wide variety of uses, such as a material for fabrication of products and as a material for packaging. Other uses of these chemicals include the production of vinyl chloride, ethylene oxide, ethylbenzene and alcohols. Hydrocarbons used as feedstock for light olefin production include natural gas, petroleum liquids, and carbonaceous materials including coal, recycled plastics or any organic material. In addition, some butylenes are generated. The effluent from a cracking unit is separated and the olefins are recovered.

Demand for olefins are increasing and other sources are becoming more important. One method of production is the direct dehydrogenation of a paraffin stream. The paraffin is dehydrogenated and the olefin is separated and recovered. However, along with this olefin generation, additional compounds are also generated which need to be removed.

### SUMMARY

The present invention provides for preparing an olefinic feedstream to an oligomerization unit having a lower aromatics content.

A first embodiment of the invention is a process for treating the feed to an oligomerization reactor comprising passing a first stream comprising paraffins having 3 or 4 carbon atoms to a dehydrogenation unit to generate a second stream comprising olefins having 3 or 4 carbon atoms; passing the second stream to first knock-out drum to generate a first liquid condensate stream and a third stream; compressing the third stream to generate a compressed third stream; washing and cooling the compressed third stream to generate a cooled compressed third stream; passing the cooled compressed third stream to a second knock-out drum to generate a second liquid condensate stream and a fourth stream; compressing the fourth stream to generate a compressed fourth stream; passing the compressed fourth stream to a fractionation unit to generate an overhead stream comprising C2 and lighter gases, and a bottoms stream comprising olefins with 3 or 4 carbon atoms; and passing the bottoms stream to an adsorber to generate an aromatics free olefin stream. An embodiment of the invention is one, any or all of prior embodiments in this paragraph up through the first embodiment in this paragraph further comprising passing the second condensate stream to a quenching unit to generate a quenched second stream and passing the quenched second stream to the first knock-out drum. An embodiment of the invention is one, any or all of prior embodiments in this paragraph up through the first embodiment in this paragraph wherein the quench fluid is an inert gas, hydrogen or a quench oil. An embodiment of the invention is one, any or all of prior embodiments in this paragraph up through the first embodiment in this paragraph wherein the adsorber comprises a molecular sieve or carbon. An embodiment of the invention is one, any or all of prior embodiments in this paragraph up through the first embodiment in this paragraph wherein the washing and cooling stage comprises contacting the third stream with a hydrocarbon wash stream to scrub out aromatics to generate a scrubbed third stream; and passing the scrubbed third stream to a cooling unit to cool the third stream and to condense hydrocarbons having 5 or more carbon atoms. An embodiment of the invention is one, any or all of prior embodiments in this paragraph up through the first embodiment in this paragraph wherein the hydrocarbon wash stream is selected from the group consisting of oligomeric distillate, hydrotreated distillate, oligomeric gasoline, debutanizer bottoms, and mixtures thereof. An embodiment of the invention is one, any or all of prior embodiments in this paragraph up through the first embodiment in this paragraph wherein the fractionation unit comprises a demethanizer to remove methane and lighter gas components from the fourth stream. The process 1 further comprising passing the second liquid condensate stream to the first knock-out drum to recover C3 or C4 hydrocarbons from the second liquid condensate stream.

A second embodiment of the invention is a process for treating an effluent stream from a dehydrogenation reactor, comprising passing the effluent stream to a quench system to generate a quenched stream; passing the quenched stream to a wash and compression system to generate a washed compressed stream; passing the washed and compressed stream to a second compression unit to generate a compressed stream; and passing the compressed stream to a fractionation unit to generate an overhead stream comprising C2 and lighter gases and a bottoms stream comprising olefins having 3 or 4 carbon atoms. An embodiment of the invention is one, any or all of prior embodiments in this paragraph up through the second embodiment in this paragraph further comprising passing the bottoms stream to an adsorber to generate an aromatics free olefin stream. An embodiment of the invention is one, any or all of prior embodiments in this paragraph up through the second embodiment in this paragraph wherein the effluent stream comprises propane and propylene or C4 olefins and paraffins. An embodiment of the invention is one, any or all of prior embodiments in this paragraph up through the second embodiment in this paragraph wherein the wash and compression system comprises the steps of passing the quenched stream to a first knock-out drum to generate a first liquid condensate stream and a second stream; passing the second stream to a compression unit to generate a third stream; washing the third stream with a wash liquid to generate a fourth stream; cooling the fourth stream to generate a cooled washed fifth stream; passing the fifth stream to a second knock-out drum to generate a second liquid condensate stream and the compressed stream. An embodiment of the invention is one, any or all of prior embodiments in this paragraph up through the second embodiment in this paragraph further comprising passing the second liquid condensate stream to the first knock-out drum to recover C3 and C4 hydrocarbons from the second liquid condensate stream. An embodiment of the invention is one, any or all of prior embodiments in this paragraph up through the second embodiment in this paragraph wherein the wash liquid is selected from the group consisting of oligomeric distillate, hydrotreated distillate, oligomeric gasoline, debutanizer bottoms, and mixtures thereof. An embodiment of the invention is one, any or all of prior embodiments in this paragraph up through the second embodiment in this paragraph further comprising passing a paraffin stream to a dehydrogenation reactor to generate the effluent stream. An embodiment of the invention is one, any or all of prior embodiments in this paragraph up through the second embodiment in this paragraph wherein the fractionation unit is a demethanizer to remove methane and lighter gas components from the fourth stream.

A third embodiment of the invention is a process for treating a quenched effluent stream from a dehydrogenation reactor, comprising passing the quenched stream to a first knock-out drum to generate a first liquid condensate stream and a second stream; passing the second stream to a compression unit to generate a third stream; washing the third stream with a wash liquid to generate a fourth stream; cooling the fourth stream to generate a cooled washed fifth stream; passing the fifth stream to a second knock-out drum to generate a second liquid condensate stream and the compressed stream; passing the washed and compressed stream to a second compression unit to generate a compressed stream; passing the compressed stream to a fractionation unit to generate an overhead stream comprising C2 and lighter gases and a bottoms stream comprising olefins having 3 or 4 carbon atoms; and passing the bottoms stream to an adsorber to generate an aromatics free olefin stream. An embodiment of the invention is one, any or all of prior embodiments in this paragraph up through the third embodiment in this paragraph further comprising passing the second liquid condensate stream to the first knock-out drum. An embodiment of the invention is one, any or all of prior embodiments in this paragraph up through the third embodiment in this paragraph wherein the wash liquid is selected from the group consisting of oligomeric distillate, hydrotreated distillate, oligomeric gasoline, debutanizer bottoms, and mixtures thereof. An embodiment of the invention is one, any or all of prior embodiments in this paragraph up through the third embodiment in this paragraph wherein the adsorber comprises a molecular sieve or carbon.

Other objects, advantages and applications of the present invention will become apparent to those skilled in the art from the following detailed description and drawings.

### BRIEF DESCRIPTION OF THE DRAWING

The FIGURE provides a schematic of the process for washing out aromatics from an olefin stream to prepare the olefin stream for passing to an oligomerization process.

### DETAILED DESCRIPTION

The production of olefins from a paraffin stream is performed through the catalytic dehydrogenation process, using a dehydrogenation reactor. The olefins can be oligomerized to form middle distillates in the C9+ range. The dehydrogenation process produces a relatively high concentration of olefins, but in addition there is the production of a small amount of aromatics as a byproduct. The aromatics include benzene, toluene, xylenes and styrene, and can be as high as 0.15 wt % of the effluent stream from the dehydrogenation reactor. The presence of the small amount of aromatics can inhibit the oligomerization of the olefins to middle distillates of C9+ compounds. The light olefins generated are from C3 to C5 olefins and are from a dehydrogenation unit for converting C3 to C5 paraffins to olefins. A feedstream to the dehydrogenation unit will be a propane stream, a butane stream, or a pentane stream.

The present invention greatly reduces the concentration of trace aromatics in the olefins stream. Trace aromatic concentrations can be significantly reduced by mixing a portion of the dehydrogenation process stream with a heavier distillate or gasoline stream to wash the aromatics out of the process stream.

The process is for treating the feed to an oligomerization reactor, as shown in the FIGURE. The process includes passing a first stream 8 comprising paraffins to a dehydrogenation unit 10. The dehydrogenation unit 10 will generate a second stream 12 comprising olefins. The second stream 12 is passed to a first knock-out drum 20, and generates a first liquid condensate stream 22 and a third stream 24. The third stream 24 is compressed to generate a compressed third stream 32. The compressed third stream 32 is washed and cooled and forms a cooled and compressed third stream 42, comprising a mixed stream of liquid and vapor. A wash fluid 34 is added to wash the compressed stream 32. The wash fluid can comprise an olefinic gasoline or distillate, and can contribute to quenching the compressed stream 32.

The cooled and compressed third stream 42 is passed to a second knock-out drum 50 to generate a second liquid condensate stream 52 and a fourth stream 54. The fourth stream 54 is compressed to generate a compressed fourth stream 62. The compressed fourth stream 62 is passed to a fractionation unit 70 to separate light components from the olefin stream. The light components comprise C2 and lighter gases and are passed out as an overhead stream 72. The fractionation unit 70 also generates a bottoms stream 74 comprising the olefins. The bottoms stream 74 is passed to an adsorber 80 to generate an aromatics free olefin stream 82. The absorber comprises a molecular sieve or carbon for adsorbing aromatic compounds.

In this embodiment, the aromatics and dienes in the oligomerization feedstocks are reduced to very low levels. The levels are further reduced by adsorption on a 13× molecular sieve, or carbon. While not intending to be limiting, other adsorbents include bentonite, activated alumina, and Y zeolites.

The second stream 12 is quenched with a quenching fluid to cool second stream 12 and condense heavier components in the second stream 12. The quenching can be performed in a quenching unit for improving contact between the quench fluid and the second stream 12 leaving the dehydrogenation unit 10.

The process can further includes passing the second condensate stream 52 to the quenching unit, using a portion of the wash fluid recovered from the cooled and compressed third stream 42. The liquid from the knock drums would be cascaded from higher pressure to lower pressure knock out drums to recover the C4 and lighter by flashing. The C3 or C4 compounds can be recovered and passed through to eventually pass out in the product stream.

The dehydrogenation unit 10 can comprise a plurality of reactors or reactor beds. The design will be dependent on the size and amount of paraffins to be dehydrogenated. The paraffins of the present invention will comprise either a propane stream or a butane stream. The olefins generate are propylene and butylenes for the respective feed streams comprising propane and butane. The olefins generated, either propylene or butylenes, are used in the generation of an olefinic gasoline or olefinic distillate.

The quench fluid can comprise a quench oil, where the quench oil is an olefinic gasoline or olefinic distillate. The quench fluid can also comprise a cooled gas, such as hydrogen or an inert gas. When the quench fluid is an inert or light gas, the quench fluid will be separated in the fractionation unit 70 and pass out in the overhead stream 72.

The washing and cooling stage 40 includes contacting the third stream 32 with a hydrocarbon wash stream 34 to scrub out aromatics from the third stream 32. The washed stream is then cooled to condense hydrocarbons having 5 or more carbon atoms generating a two phase cooled and washed stream 42. The cooled and washed stream will separate the liquid from the vapor in a knock-out drum 50.

The wash stream 34 comprises a hydrocarbon stream for absorbing aromatics. The wash stream preferably is selected from hydrocarbon streams comprising oligomeric distillate, hydrotreated distillate, oligomeric gasoline, debutanizer bottoms, or mixtures of the above hydrocarbon streams.

Benzene and heavier aromatics are scrubbed out of the vapor product by washing the compressor intercoolers with oligomerization distillate or gasoline product. Since the benzene and heavier aromatics can comprise a concentration of 0.15 wt % in reactor effluent, washing the compressor intercoolers with 2 wt % of oligomer product would remove 90-95% of the aromatics.

In one embodiment, the fractionation unit comprises a demethanizer to remove methane and lighter gases from the fourth stream.

The process can be continuous, and the adsorber 80 will periodically need to be regenerated. The process can include two or more adsorbers, with at least one adsorber off-line. The off-line adsorber is regenerated and brought on-line when an on-line adsorber has reached capacity for removing aromatics. The off-line adsorber in regeneration mode is regenerated by passing a regenerant stream through the adsorber to displace or desorb the adsorbed aromatics and dienes. The regenerant stream can be nitrogen or a slipstream of product LPG (liquefied petroleum gas), gasoline or distillate. Oligomeric gasoline may be the preferred regenerant as the recovered aromatics would be routed to gasoline.

The liquid from the lowest pressure knock-out drum will have the highest concentration of aromatics and is removed from the knock-out drum. This liquid is passed to a suitable process unit in the dehydrogenation plant for recovery of the oligomer, either propylene or butylenes. Suitable process units would include the stabilizer of the hydrotreater, the light olefin oligomerization debutanizer, or the light olefin oligomerization product splitter. The product splitter would separate the aromatics from the product stream and the aromatics would be passed to the olefinic gasoline byproduct and wash liquid to the distillate product.

While the invention has been described with what are presently considered the preferred embodiments, it is to be understood that the invention is not limited to the disclosed embodiments, but it is intended to cover various modifications and equivalent arrangements included within the scope of the appended claims.

What is claimed is:

1. A process for treating the feed to an oligomerization reactor comprising:
passing a first stream comprising paraffins having 3 or 4 carbon atoms to a dehydrogenation unit to generate a second stream comprising olefins having 3 or 4 carbon atoms;
passing the second stream to first knock-out drum to generate a first liquid condensate stream and a third stream;
compressing the third stream to generate a compressed third stream;
washing and cooling the compressed third stream to generate a cooled compressed third stream;
passing the cooled compressed third stream to a second knock-out drum to generate a second liquid condensate stream and a fourth stream;
compressing the fourth stream to generate a compressed fourth stream;
passing the compressed fourth stream to a fractionation unit to generate an overhead stream comprising C2 and lighter gases, and a bottoms stream comprising olefins with 3 or 4 carbon atoms; and
passing the bottoms stream to an adsorber to generate an aromatics free olefin stream.

2. The process of claim 1 further comprising passing the second condensate stream to a quenching unit to generate a quenched second stream and passing the quenched second stream to the first knock-out drum.

3. The process of claim 2 wherein the quench fluid is an inert gas, hydrogen or a quench oil.

4. The process of claim 1 wherein the adsorber comprises a molecular sieve or carbon.

5. The process of claim 1 wherein the washing and cooling stage comprises:
contacting the third stream with a hydrocarbon wash stream to scrub out aromatics to generate a scrubbed third stream; and
passing the scrubbed third stream to a cooling unit to cool the third stream and to condense hydrocarbons having 5 or more carbon atoms.

6. The process of claim 5 wherein the hydrocarbon wash stream is selected from the group consisting of oligomeric distillate, hydrotreated distillate, oligomeric gasoline, debutanizer bottoms, and mixtures thereof.

7. The process of claim 1 wherein the fractionation unit comprises a demethanizer to remove methane and lighter gas components from the fourth stream.

8. The process 1 further comprising passing the second liquid condensate stream to the first knock-out drum to recover C3 or C4 hydrocarbons from the second liquid condensate stream.

9. A process for treating an effluent stream from a dehydrogenation reactor, comprising:
passing the effluent stream to a quench system to generate a quenched stream;
passing the quenched stream to a wash and compression system to generate a washed compressed stream;
passing the washed and compressed stream to a second compression unit to generate a compressed stream; and
passing the compressed stream to a fractionation unit to generate an overhead stream comprising C2 and lighter gases and a bottoms stream comprising olefins having 3 or 4 carbon atoms.

10. The process of claim 9 further comprising passing the bottoms stream to an adsorber to generate an aromatics free olefin stream.

11. The process of claim 9 wherein the effluent stream comprises propane and propylene or C4 olefins and paraffins.

12. The process of claim 9 wherein the wash and compression system comprises the steps of:
passing the quenched stream to a first knock-out drum to generate a first liquid condensate stream and a second stream;
passing the second stream to a compression unit to generate a third stream;
washing the third stream with a wash liquid to generate a fourth stream;
cooling the fourth stream to generate a cooled washed fifth stream;
passing the fifth stream to a second knock-out drum to generate a second liquid condensate stream and the compressed stream.

13. The process of claim 12 further comprising passing the second liquid condensate stream to the first knock-out drum to recover C3 and C4 hydrocarbons from the second liquid condensate stream.

14. The process of claim 12 wherein the wash liquid is selected from the group consisting of oligomeric distillate, hydrotreated distillate, oligomeric gasoline, debutanizer bottoms, and mixtures thereof.

15. The process of claim 9 further comprising passing a paraffin stream to a dehydrogenation reactor to generate the effluent stream.

16. The process of claim 9 wherein the fractionation unit is a demethanizer to remove methane and lighter gas components from the fourth stream.

17. A process for treating a quenched effluent stream from a dehydrogenation reactor, comprising:
passing the quenched stream to a first knock-out drum to generate a first liquid condensate stream and a second stream;
passing the second stream to a compression unit to generate a third stream;
washing the third stream with a wash liquid to generate a fourth stream;
cooling the fourth stream to generate a cooled washed fifth stream;
passing the fifth stream to a second knock-out drum to generate a second liquid condensate stream and the compressed stream;
passing the washed and compressed stream to a second compression unit to generate a compressed stream;
passing the compressed stream to a fractionation unit to generate an overhead stream comprising C2 and lighter gases and a bottoms stream comprising olefins having 3 or 4 carbon atoms; and
passing the bottoms stream to an adsorber to generate an aromatics free olefin stream.

18. The process of claim 17 further comprising passing the second liquid condensate stream to the first knock-out drum.

19. The process of claim 17 wherein the wash liquid is selected from the group consisting of oligomeric distillate, hydrotreated distillate, oligomeric gasoline, debutanizer bottoms, and mixtures thereof.

20. The process of claim 17 wherein the adsorber comprises a molecular sieve or carbon.
